(19) United States
(12) Patent Application Publication
(10) Pub. No.: US 2016/0296511 A1
(43) Pub. Date: Oct. 13, 2016

(54) LAQUINIMOD FOR REDUCING THALAMIC DAMAGE IN MULTIPLE SCLEROSIS

(71) Applicants: Massimo Filippi, Cambiago (MI) (IT); Giancarlo Comi, Carvico (BG) (IT); Maria Assunta Rocca, Dolzago (LC) (IT)

(72) Inventors: Massimo Filippi, Cambiago (MI) (IT); Giancarlo Comi, Carvico (BG) (IT); Maria Assunta Rocca, Dolzago (LC) (IT)

(73) Assignee: Teva Pharmaceutical Industries, Ltd., Petach-Tikva (IL)

(21) Appl. No.: 15/186,163

(22) Filed: Jun. 17, 2016

Related U.S. Application Data
(63) Continuation of application No. 14/049,411, filed on Oct. 9, 2013.
(60) Provisional application No. 61/713,256, filed on Oct. 12, 2012.

(51) Int. Cl. A61K 31/4704; A61K 9/00

(52) U.S. Cl. CPC A61K 31/4704; A61K 9/0053

(57) ABSTRACT

This invention provides methods for inhibiting or reducing thalamic damage in a subject comprising administering to the subject an amount of laquinimod, wherein the subject is a human patient afflicted with a form of multiple sclerosis or presenting a clinically isolated syndrome who has been determined to have thalamic damage at baseline, a subject afflicted with a disease or disorder other than a form of multiple sclerosis or a clinically isolated syndrome, or a subject not afflicted with a form of multiple sclerosis or a presenting clinically isolated syndrome, and laquinimod and laquinimod pharmaceutical compositions for use thereof. This invention also provides methods for inhibiting or reducing tremor or spasticity in a subject afflicted by tremor or spasticity, comprising administering to the subject an amount of laquinimod, and laquinimod and laquinimod pharmaceutical compositions for use thereof.

This application claims benefit of U.S. Provisional Application No. 61/713,256, filed Oct. 12, 2012, the entire content of which is hereby incorporated by reference herein.

Throughout this application, various publications are referred to by first author and year of publication. Full citations for these publications are presented in a References section immediately before the claims. Disclosures of the documents and publications cited and those in the References section are hereby incorporated by reference in their entireties into this application in order to more fully describe the state of the art as of the date of the invention described herein.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

It is generally assumed that MS is mediated by some kind of autoimmune process possibly triggered by infection and superimposed upon a genetic predisposition. It is a chronic inflammatory condition that damages the myelin of the Central Nervous System (CNS). The pathogenesis of MS is characterized by the infiltration of autoreactive T-cells from the circulation directed against myelin antigens into the CNS (Bjartmar, 2002). In addition to the inflammatory phase in MS, axonal loss occurs early in the course of the disease and can be extensive over time, leading to the subsequent development of progressive, permanent, neurologic impairment and, frequently, severe disability (Neuhaus, 2003). Symptoms associated with the disease include fatigue, spasticity, ataxia, weakness, bladder and bowel disturbances, sexual dysfunction, pain, tremor, paroxysmal manifestations, visual impairment, psychological problems and cognitive dysfunction (EMEA Guideline, 2006).

MS disease activity can be monitored by magnetic resonance imaging (MRI) of the brain, accumulation of disability, as well as rate and severity of relapses. The diagnosis of clinically definite MS as determined by the Poser criteria (Poser, 1983) requires at least two neurological events suggesting demyelination in the CNS separated in time and in location. A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite MS (CDMS). Over 80 percent of patients with a CIS and MRI lesions go on to develop MS, while approximately 20 percent have a self-limited process (Brex, 2002; Frohman, 2003).

Various MS disease stages and/or types are described in Multiple Sclerosis Therapeutics (Duntiz, 1999). Among them, relapsing-remitting MS (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination (Neuhaus, 2003; Noseworthy, 2000).

In April 2001, an international panel in association with the National MS Society of America recommended diagnostic criteria for MS. These criteria became known as the McDonald Criteria. The McDonald Criteria make use of MRI techniques and are intended to replace the Poser Criteria and the older Schumacher Criteria (McDonald, 2001). The McDonald Criteria was revised in March 2005 by an international panel (Polman, 2005) and updated again in 2010 (Polman, 2011).

Intervention with disease-modifying therapy at relapsing stages of MS is suggested to reduce and/or prevent accumulating neurodegeneration (Hohlfeld, 2000; De Stefano, 1999). There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and fingolimod (Gilenya®). Most of them are believed to act as immunomodulators. Mitoxantrone and natalizumab are believed to act as immunosuppressants. However, the mechanisms of action of each have been only partly elucidated. Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

### Thalamus and Thalamic Damage

The human thalamus is a nuclear complex located in the diencephalon and comprising of four parts, the hypothalamus, the epythalamus, the ventral thalamus, and the dorsal thalamus. The thalamus is a relay centre subserving both sensory and motor mechanisms. Thalamic nuclei (50-60 nuclei) projects to one or a few well-defined cortical areas. Multiple cortical areas receive afferents from a single thalamic nucleus and send back information to different thalamic nuclei. The corticofugal projection provides positive feedback to the “correct” input, while at the same time suppressing irrelevant information. Topographical organization of the thalamic afferents and efferents is contralateral, and the lateralization of the thalamic function affects both sensory and motoric aspects. Symptoms of lesions located in the thalamus are closely related to the function of the areas involved. An infarction or haemorrhage thalamic lesion and develop somatosensory disturbances and/or central pain in the opposite hemibody, analgesic or purely algesic thalamic syndrome characterized by contralateral anaesthesia (or hypaesthesia), contralateral weakenss, ataxia and, often, persistent spontaneous pain (Trinidad Herrero, 2002). Other diseases and conditions which have been associated with damage to the thalamus include movement disorders, dystonia, athetosis, chorea, tremor, jerky, myoclonic movements, involuntary movements, ataxia, pain, tremor, spasticity Alzheimer's disease, Huntington's disease, MS and Dejerine-Roussy syndrome (thalamic pain syndrome) (Kim, 2001; Jong, 2008; Kassubek, 2005; Tuling, 1999; Lee, 1994; Sheline, 2003, Torres, 2010; Stachowiak, 2007).

### Laquinimod

Laquinimod is a novel synthetic compound with high oral bioavailability which has been suggested as an oral formulation for the treatment of MS (Polman, 2005; Sandberg-Wollheim, 2005). Laquinimod and its sodium salt form are described, for example, in U.S. Pat. No. 6,077,851.

The mechanism of action of laquinimod is not fully understood. Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFκB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runström, 2006; Brick, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

### SUMMARY OF THE INVENTION

This invention provides a method for inhibiting or reducing thalamic damage in a subject afflicted with a form of MS or presenting a CIS, comprising orally administering to the subject an amount of laquinimod so as to thereby inhibit or reduce thalamic damage in the subject, wherein the subject is a human patient who has been determined to have thalamic damage at baseline.

This invention also provides a method for inhibiting or reducing thalamic damage in a subject afflicted with a disease or disorder other than a form of MS or a CIS, comprising administering to the subject an amount of laquinimod so as to thereby inhibit or reduce thalamic damage in the subject.

This invention also provides a method for inhibiting or reducing tremor or spasticity in a subject afflicted by tremor or spasticity, comprising administering to the subject an amount of laquinimod so as to thereby inhibit or reduce the tremor or the spasticity in the subject.

This invention also provides laquinimod for use in inhibiting or reducing thalamic damage in a human patient who has been determined to have thalamic damage at baseline.

This invention also provides a pharmaceutical composition comprising an amount of laquinimod for use in inhibiting or reducing thalamic damage in a human patient who has been determined to have thalamic damage at baseline.

This invention also provides laquinimod for use in inhibiting or reducing thalamic damage in a subject afflicted with a disease or disorder other than a form of MS or a CIS.

This invention also provides a pharmaceutical composition comprising an amount of laquinimod for use in inhibiting or reducing thalamic damage in a subject afflicted with a disease or disorder other than a form of MS or a CIS.

This invention also provides laquinimod for use in inhibiting or reducing thalamic damage in a subject not afflicted with a form of MS or presenting a CIS.

This invention also provides a pharmaceutical composition comprising an amount of laquinimod for use in inhibiting or reducing thalamic damage in a subject not afflicted with a form of MS or presenting a CIS.

This invention also provides laquinimod for use in inhibiting or reducing tremor or spasticity in a subject.

This invention also provides a pharmaceutical composition comprising an amount of laquinimod for use in inhibiting or reducing tremor or spasticity in a subject.

### BRIEF DESCRIPTION OF THE DRAWINGS

FIG. 1: FIG. 1 is a graph of Patient Disposition from Example 2. (*For technical reasons, baseline and/or post-baseline scans from two patients in the laquinimod arm and three patients in the placebo arm were not evaluable. These patients were excluded from this analysis.)

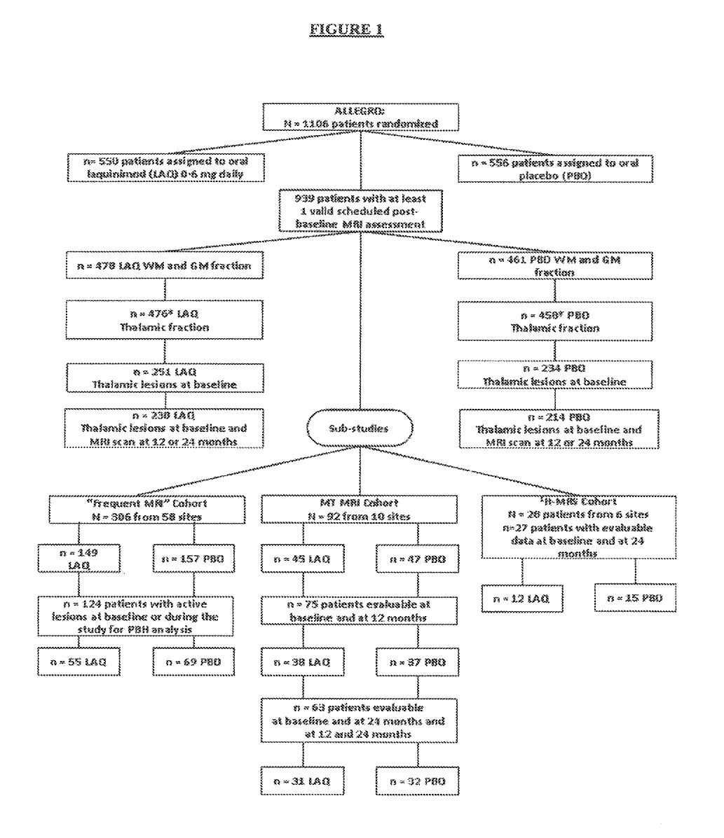

### DETAILED DESCRIPTION OF THE INVENTION

This invention provides a method for inhibiting or reducing thalamic damage in a subject afflicted with a form of MS or presenting a CIS, comprising orally administering to the subject an amount of laquinimod so as to thereby inhibit or reduce thalamic damage in the subject, wherein the subject is a human patient who has been determined to have thalamic damage at baseline.

In one embodiment of the present invention, the form of MS is RRMS. In another embodiment, of the present invention, the form of MS is a progressive form of MS.

In one embodiment, the patient is a naïve patient. In another embodiment, the patient has previously received at least one MS therapy.

In an embodiment, the subject has been determined to have at least one thalamic lesion at baseline. In another embodiment, the thalamic lesion is a T2 thalamic lesion

This invention also provides a method for inhibiting or reducing thalamic damage in a subject afflicted with a disease or disorder other than a form of MS or a CIS, comprising administering to the subject an amount of laquinimod so as to thereby inhibit or reduce thalamic damage in the subject.

In one embodiment of the present invention, the subject is a human. In another embodiment, the subject is not afflicted with a form of MS and is not presenting a CIS. In yet another embodiment, the subject is a naïve subject.

In one embodiment, the subject is afflicted with a condition or disorder which is associated with thalamic damage. In another embodiment, the subject is afflicted with dystonia, athetosis, chorea, tremor, jerky, myoclonic movements, involuntary movements, ataxia, pain, tremor or spasticity.

In an embodiment, the subject is afflicted with a movement disorder and the administration of laquinimod is effective to treat the subject. In another embodiment, the movement disorder is dystonia, paroxysmal dystonia, asterixis, chorea, ballism-chorea, myorhythmic movements, dyskinesia, bepharospasm, ataxia, epilepsy, seizures or convulsions.

In an embodiment, the subject is afflicted with a mood disorder and the administration of laquinimod is effective to treat the subject. In another embodiment, the mood disorder is depression, anxiety or bipolar disorder.

In an embodiment, the subject is afflicted with Parkinson's disease, Alzheimer's disease, schizophrenia or Huntington's disease and the administration of laquinimod is effective to treat the subject. In another embodiment, the subject is afflicted with thalamic pain syndrome and the administration of laquinimod is effective to treat the subject.

In one embodiment, the subject has been determined to have thalamic damage at baseline. In another embodiment, the thalamic damage is a thalamic lesion. In another embodiment, the thalamic lesion is a T2 thalamic lesion In another embodiment, the thalamic damage is measured using MRI.

In one embodiment, the subject is afflicted by tremor or spasticity. In another embodiment, the subject is a human patient diagnosed to be afflicted by tremor or spasticity. In another embodiment, the subject is diagnosed to be afflicted by tremor or spasticity which is treatable by laquinimod. In another embodiment, the administration of laquinimod is effective to reduce or inhibit tremor in the subject. In another embodiment, the administration of laquinimod is effective to reduce or inhibit spasticity in the subject. In yet another embodiment, the subject has previously suffered a thalamic stroke.

In one embodiment, laquinimod is administered via oral administration. In another embodiment, laquinimod is administered periodically. In another embodiment, the periodic administration is for a period of greater than 24 weeks. In another embodiment, laquinimod is administered daily. In another embodiment, laquinimod is administered more often than once daily. In another embodiment, laquinimod is administered less often than once daily.

In one embodiment, the amount laquinimod administered is 0.1-2.5 mg/day. In another embodiment, the amount laquinimod administered is 0.25-2.0 mg/day. In another embodiment, the amount laquinimod administered is 0.3-0.9 mg/day. In another embodiment, the amount laquinimod administered is 0.5-1.2 mg/day. In another embodiment, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day. In another embodiment, the amount laquinimod administered is 1.0 mg/day. In another embodiment, the amount laquinimod administered is 1.2 mg/day. In another embodiment, the amount laquinimod administered is 1.5 mg/day. In yet another embodiment, the amount laquinimod administered is 2.0 mg/day.

This invention also provides a method for inhibiting or reducing tremor or spasticity in a subject afflicted by tremor or spasticity, comprising administering to the subject an amount of laquinimod so as to thereby inhibit or reduce the tremor or the spasticity in the subject.

In one embodiment, the subject is a human patient afflicted with a form of MS or presenting a CIS. In another embodiment, the subject is a human patient not afflicted with a form of MS or presenting a CIS. In another embodiment, the subject is a human patient diagnosed to be afflicted by tremor or spasticity. In another embodiment, the subject is afflicted by tremor or spasticity which is treatable by laquinimod.

In one embodiment, the subject has been determined to have thalamic damage at baseline. In another embodiment, the thalamic damage is a thalamic lesion. In another embodiment, the thalamic lesion is a T2 thalamic lesion In another embodiment, the thalamic damage is measured using MRI.

This invention also provides laquinimod for use in inhibiting or reducing thalamic damage in a human patient who has been determined to have thalamic damage at baseline.

This invention also provides a pharmaceutical composition comprising an amount of laquinimod for use in inhibiting or reducing thalamic damage in a human patient who has been determined to have thalamic damage at baseline.

This invention also provides laquinimod for use in inhibiting or reducing thalamic damage in a subject afflicted with a disease or disorder other than a form of MS or a CIS.

This invention also provides a pharmaceutical composition comprising an amount of laquinimod for use in inhibiting or reducing thalamic damage in a subject afflicted with a disease or disorder other than a form of MS or a CIS.

This invention also provides laquinimod for use in inhibiting or reducing thalamic damage in a subject not afflicted with a form of MS or presenting a CIS.

This invention also provides a pharmaceutical composition comprising an amount of laquinimod for use in inhibiting or reducing thalamic damage in a subject not afflicted with a form of MS or presenting a CIS.

This invention also provides laquinimod for use in inhibiting or reducing tremor or spasticity in a subject.

This invention also provides a pharmaceutical composition comprising an amount of laquinimod for use in inhibiting or reducing tremor or spasticity in a subject.

For the foregoing embodiments, each embodiment disclosed herein is contemplated as being applicable to each of the other disclosed embodiments. In addition, the elements recited in the packaging and pharmaceutical composition embodiments can be used in the method and use embodiments described herein.

### Laquinimod

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in their entireties into this application.

Use of laquinimod for treatment of various conditions, and the corresponding dosages and regimens, are described in U.S. Pat. No. 6,077,851 (MS, insulin-dependent diabetes mellitus, systemic lupus erythematosus, rheumatoid arthritis, inflammatory bowel disease, psoriasis, inflammatory respiratory disorder, atherosclerosis, stroke, and Alzhemier's disease), U.S. Application Publication No. 2011-0027219 (Crohn's disease), U.S. Application Publication No. 2010-0322900 (Relapsing-remitting multiple sclerosis), U.S. Application Publication No. 2011-0034508 (brain-derived neurotrophic factor (BDNF)-related diseases), U.S. Application Publication No. 2011-0218179 (active lupus nephritis), U.S. Application Publication No. 2011-0218203 (rheumatoid arthritis), U.S. Application Publication No. 2011-0217295 (active lupus arthritis), and U.S. Application Publication No. 2012-0142730 (reducing fatigue, improving quality of life, and providing neuroprotection in MS patients), each of which is hereby incorporated by reference in their entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit can be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators (disintegrants) include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248. These references in their entireties are hereby incorporated by reference into this application.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is the use of laquinimod for inhibiting or reducing thalamic damage in a subject, particularly a subject who has thalamic damage. The ability of laquinimod to inhibit or reduce thalamic damages was not previously disclosed. In addition, a method of inhibiting or reducing tremor and spasticity in a subject using laquinimod is disclosed. Previously, it was not known that laquinimod can inhibit or reduce tremor or spasticity in a subject.

### TERMS

As used herein, and unless stated otherwise, each of the following terms shall have the definition set forth below.

As used herein, “laquinimod” means laquinimod acid or a pharmaceutically acceptable salt thereof.

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, “about” in the context of a numerical value or range means±10% of the numerical value or range recited or claimed.

As used herein, “effective” when referring to an amount of laquinimod and/or pridopidine refers to the quantity of laquinimod that is sufficient to yield a desired therapeutic response without undue adverse side effects (such as toxicity, irritation, or allergic response) commensurate with a reasonable benefit/risk ratio when used in the manner of this invention.

“Administering to the subject” or “administering to the (human) patient” means the giving of, dispensing of, or application of medicines, drugs, or remedies to a subject/patient to relieve, cure, or reduce the symptoms associated with a disease, disorder or condition, e.g., a pathological condition.

“Treating” as used herein encompasses, e.g., inducing inhibition, regression, or stasis of a disease or disorder, or lessening, suppressing, inhibiting, reducing the severity of, eliminating or substantially eliminating, or ameliorating a symptom of the disease or disorder.

“Inhibition” of disease progression or disease complication in a subject means preventing or reducing the disease progression and/or disease complication in the subject.

A “symptom” associated with a disease or disorder includes any clinical or laboratory manifestation associated with the disease or disorder and is not limited to what the subject can feel or observe.

As used herein, “a subject afflicted with” a disease, disorder or condition means a subject who has been clinically diagnosed to have the disease, disorder or condition.

As used herein, a subject at “baseline” is as subject prior to administration of laquinimod.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### Experimental Details

In phase III ALLEGRO and BRAVO clinical trials of oral laquinimod it was demonstrated that laquinimod at 0.6 mg/day slowed disability and brain atrophy progression in RRMS patients, suggesting that the drug may have neuroprotective in addition to anti-inflammatory effects (U.S. Application Publication No. 2012-0142730; Comi, 2012; Vollmer, 2011). As a small molecule, laquinimod enters the CNS and interacts with resident inflammatory cells, including microglia, astrocytes, and oligodendrocytes. Laquinimod is thought to reduce astrocyte activation induced by pro-inflammatory cytokines without causing immunosuppression (Wegner, 2010; Bruck, 2012).

Both the ALLEGRO and the BRAVO trial showed that laquinimod decreased the rate of whole brain atrophy compared with placebo (Comi, 2012; Vollmer 2011). However, it was not determined whether protection from tissue damage is diffuse or limited to a specific brain compartment.

A summary of the ALLEGRO trial is provided in Example 1 below:

### Example 1

### Clinical Trial (Phase III)—Assessment of Oral Laquinimod in Preventing Progression of MS

A multinational (24 countries), multicenter (approximately 139 sites), randomized, double-blinded, parallel-group, placebo-controlled clinical trial (“ALLEGRO” or MS-LAQ-301) was conducted to evaluate the efficacy, safety and tolerability of daily oral administration of laquinimod 0.6 mg in subjects with RRMS for a 24 months duration.

One thousand one hundred and six (1106) patients were equally randomized to either laquinimod 0.6 mg or placebo and treated in a double-blind manner and baseline characteristics were balanced between groups. The primary endpoint of the study was the number of confirmed relapses during the double-blind treatment period, which corresponds to the annualized relapse rate (ARR—number of relapses divided by total exposure of all patients). Secondary endpoints included disability as measured by Expanded Disability Status Scale (EDSS) changes confirmed at 3 months, and cumulative number of gadolinium enhancing (GdE) and new/enlarging T2 MRI lesions.

### Study Duration

### Screening Phase: 1 Month.

Double blind treatment phase: 24 months of once-daily oral administration of daily dose of 0.6 mg laquinimod or matching placebo.

Upon blinded variance and power reassessment of the population progression (planned prior to first subject completes the 20 months of treatment), the double blind study duration may be extended to 30 months. This is planned in order to enhance the statistical power to detect the effect of laquinimod on disability accumulation. The recommendation to extend the study duration is based on a pre-defined rule.

### Study Design

Eligible subjects were equally randomized 1:1 into one of the following treatment arms:
1. Laquinimod capsules 0.6 mg: One 0.6 mg laquinimod capsule was administered orally once daily. The 0.6 mg laquinimod capsules contain 0.6 mg of Laquinimod Acid per capsule with meglumine, and were manufactured according to the method disclosed in PCT International Application Publication No. WO/2007/146248, published Dec. 21, 2007 (see, page 10, line 5 to page 11, line 3).
2. Matching placebo for laquinimod arm: one capsule is administered once daily.

Subjects were evaluated at study sites for 12 scheduled visits of the double blind phase at months: −1 (screening), 0 (baseline), 1, 2, 3, 6, 9, 12, 15, 18, 21 and 24 (termination/early discontinuation). In case of the 6 months extended study, subjects were evaluated at study sites at months 27 and 30 (termination/early discontinuation of extended study), in this case month 24 was a regular scheduled visit.

EDSS was assessed every 3 months, MSFC every 6 months, and MRI was performed annually in all patients. A subgroup of patients (n=189) underwent additional MRI scans at months 3 and 6. Subjects successfully completing the study were offered the opportunity to enter into a 1-year open label extension. Patients who discontinued the study underwent a final termination visit and were not further evaluated, except for those who discontinued due to adverse events.

The following assessments were performed at specified time points:
1. Vital signs were measured at each study visit.
2. A physical examination is performed at months −1 (screening), 0 (baseline) 1, 3, 6, 12, 18 and 24 (termination/early discontinuation core study). In case of the 6 months extended study, additional examination was performed at month 30 (termination/early discontinuation of extended study).
3. The following safety clinical laboratory tests were performed:
   a. Complete blood count (CBC) with differential—at all scheduled visits. A reticulocyte count was added to the CBC at months 0 (baseline) and 24/30 (termination/early discontinuation).
   b. Serum chemistry (including electrolytes, liver enzymes, direct and total bilirubin and pancreatic amylase and CPK), and urinalysis—at all scheduled visits.
   c. A rapid urine β-hCG test was performed in women of child-bearing potential at baseline (month 0) and at each scheduled study visit thereafter (at site).
   d. β-hCG in women of child-bearing potential was performed at all scheduled visits.
   e. Starting after visit Month 3 a rapid urine β-hCG test was performed in women of child-bearing potential every 28 (±2) days. The subject was contacted by telephone within 72 hours after the test was scheduled to be performed and asked specific questions regarding the test. In case of suspected pregnancy (positive urine β-hCG test result), the caller made sure that the study drug has been discontinued and the subject was instructed to arrive at the site as soon as possible with all study drugs.
4. Markers of inflammation (serum conventional C-reactive protein and fibrinogen)—at screening, baseline and all scheduled visits thereafter.
5. During the first 3 months periodical phone calls were placed by the site personnel every two weeks. A list of predefined questions relating to signs/symptoms suggestive of vascular thrombosis was presented to the subjects.
6. ECG was performed at months −1 (screening; additional recording, up to 30 minutes apart is performed if QT_{c} is less than 450 msec), (baseline; three recordings, 15 minutes apart), 1, 2, 3, 6, 12, 18 and 24 (termination/early discontinuation). In case of the 6 months extended study, ECG is performed at month 30 (termination/early discontinuation of the extended study).
7. Chest X-ray is performed at months −1 (screening), (if not performed within 7 months prior to the screening visit).
8. Adverse Events (AEs) are monitored throughout the study.
9. Concomitant medications are monitored throughout the study.
10. Neurological evaluations, including Expanded Disability Status Scale (EDSS), 25 foot walk test/Ambulation Index (AI), Functional systems (FS) are performed at months −1 (screening), 0 (baseline) and every 3 months during the study and the extended study period.
11. MS functional Composite (MSFC) was assessed at months −1 (screening) (three practices for training purposes only), at month 0 (baseline), 6, 12, 18 and 24 (termination/early discontinuation). In case of the 6 months extended study, the last MSFC was performed at months 30 (termination/early discontinuation of the extended study).
12. Subject-reported fatigue was assessed by the Modified Fatigue Impact Scale (MFIS) at months 0, 6, 12, 18, and 24 (termination/early discontinuation). In case of the 6 months extended study, additional MFIS was performed at month 30 (termination/early discontinuation of the extended study).
13. The general health status was assessed by the EuroQoL (EQ5D) questionnaire at month 0 (baseline) and month 24 (termination/early discontinuation of the study). In case of the 6 months extended study, the last EuroQoL (EQ5D) was performed at month 30 (termination/early discontinuation of the extended study) instead of month 24.
14. The general health status was assessed by the Short-Form general health survey (SF-36) subject-reported questionnaire at month 0 (baseline) and every 6 months thereafter, until termination/early discontinuation.
15. The subject underwent 5 assessments of binocular low-contrast visual acuity using the 100%, 2.5% and 1.25% contrast level charts [Sloan letter or Tumbling-E] in each assessment, at months 0 (baseline), 6, 12, 18 and 24 (termination/early discontinuation). In case of extending the study for 6 months, additional binocular low-contrast visual acuity assessment is performed at month 30 (termination/early discontinuation of the extended study).
16. Serum samples were collected from all subjects in order to investigate the potential mechanism of action of laquinimod and additional biomarkers of inflammation and potential biomarkers of MS disease at months: 0, 1, 12 and 24. In case of extending the study for 6 months the last serum sample is performed at month 30 (termination/early discontinuation of the extended study) instead of month 24.
17. The subjects underwent 3 MRI scans at months 0 (baseline), 12 and 24 (termination/early discontinuation). In case of the 6 months extended study, an additional MRI was performed at month 30 (termination/early discontinuation of the extended study).
18. Population PK study (PPK): Blood samples for PPK evaluation were collected from all subjects at months 1, 12 and 24. In case of extending the study for 6 months the last PPK evaluation was performed at month 30 (termination/early discontinuation of the extended study) instead of month 24.
19. Relapses were confirmed/monitored through the study. Since the “in study” relapse definition must be supported by an objective neurological evaluation, a neurological deficit must sustain long enough to eliminate pseudo-relapses. Therefore, in this clinical trial, a relapse was the appearance of one or more new neurological abnormalities or the reappearance of one or more previously observed neurological abnormalities wherein the change in clinical state lasts at least 48 hours and is immediately preceded by an improving neurological state of at least thirty (30) days from onset of previous relapse.
20. The allowed treatment for a relapse was intravenous Methylprednisolone 1 gr/day for up to 5 consecutive days.

### Re-Consent Criteria

Upon a confirmed diagnosis of MS relapse, (as defined in the protocol) or an increase in EDSS in 12.0 points, sustained for 23 months, the following actions were taken:
1. The subject was reminded of the current available MS medications and the opportunity to terminate the study as written in the informed consent form.
2. The subject was requested to re-sign an informed consent form if he/she chooses to continue to participate in the study, in the same treatment assignment.

Safety stopping rules were set in place for the management of: 1) elevated liver enzymes, 2) inflammatory events, 3) thrombotic events and 4) pancreatitis.

### Ancillary Studies:

1. Frequent MRI (selected countries and sites only): The cumulative number of T₁-Gd enhancing lesions taken from scans obtained at months 0, 3, 6, 12, and 24, and in case the study is be extended, 30. Additional MRIs for the ancillary study are performed at months 3 and 6.
2. Magnetization Transfer (MT) (selected countries and sites only): the change from baseline to month 12 and 24/30 months in magnetization transfer MRI. MT was assessed at months 0 (baseline), 12 and 24 (termination/early discontinuation). In case of the 6 months extended study, the last MT was performed at month 30 (termination/early discontinuation of the extended study) instead of month 24.
3. Proton MR Spectroscopy (¹H-MRS) (selected countries and sites only): Change from baseline to 24/30 in ¹H-MRS metabolites. ¹H-MRS was assessed at months 0 (baseline), and 24 (termination/early discontinuation). In case of the 6 months extended study, the last ¹H-MRS was performed at month 30 (termination/early discontinuation of the extended study) instead of month 24.
4. Pharmacogenetic (PGx) assessment: Blood samples for PGx parameters were collected from all subjects at screening.
5. Brain atrophy, as defined by the percentage of change from one scan to the subsequent scan in brain volume, in addition to the measurements done in the main study (Frequent MRI Cohort).
6. Whole blood and serum samples (selected countries and sites only) were collected for evaluation of the immunological response to treatment with laquinimod and further investigation of the potential mechanism of action. Whole blood samples were collected at months: 0, 1, 3, 6, 12 and 24. Serum samples were collected at month: 0, 1, 6, 12 and 24 (even if the study is extended to month 30).
7. Relationship between PGx and response to laquinimod in terms of clinical, MRI and safety parameters.

### Inclusion/Exclusion Criteria

### Inclusion Criteria

1. Subjects must have a confirmed and documented diagnosis as defined by the Revised McDonald Criteria (Polman, 2005), with relapsing-remitting disease course.
2. Subjects must be ambulatory with converted Kurtzke EDSS score of 0-5.5.
3. Subjects must be in a stable neurological condition and free of corticosteroid treatment [intravenous (iv), intramuscular (im) and/or per os (po)]30 days prior to screening (month −1).
4. Subjects must have experienced one of the following:
   a. At least one documented relapse in the 12 months prior to screening.
   b. At least two documented relapses in the 24 months prior to screening.
   c. One documented relapse between 12 and 24 months prior to screening with at least one documented T1-Gd enhancing lesion in an MRI performed within 12 months prior to screening.
5. Subjects must be between 18 and 55 years of age, inclusive.
6. Subjects must have disease duration of at least 6 months (from the first symptom) prior to screening.
7. Women of child-bearing potential must practice an acceptable method of birth control. Acceptable method of birth control in this study include: surgical sterilization, intrauterine devices, oral contraceptive, contraceptive patch, long-acting injectable contraceptive, partner's vasectomy or double barrier method (condom or diaphragm with spermicide).
8. Subjects must be able to sign and date a written informed consent prior to entering the study.
9. Subjects must be willing and able to comply with the protocol requirements for the duration of the study.

### Exclusion Criteria

1. Subjects with progressive forms of MS.
2. An onset of relapse, unstable neurological condition or any treatment with corticosteroids [(iv), intramuscular (im) and/or per os (po)] or ACTH between months −1 (screening) and 0 (baseline).
3. Use of experimental or investigational drugs, and/or participation in drug clinical studies within the 6 months prior to screening.
4. Use of immunosuppressive including mitoxantrone (Novantrone®) or cytotoxic agents within 6 months prior to screening visit.
5. Previous use of any one of the following: natalizumab (Tysabri®), cladribine, laquinimod.
6. Previous treatment with glatiramer acetate (Copaxone®) Interferon-β (either 1a or 1b) or intravenous immunoglobulin (IVIG) within 2 months prior to screening visit.
7. Systemic corticosteroid treatment of ≧30 consecutive days duration within 2 months prior to screening visit.
8. Previous total body irradiation or total lymphoid irradiation.
9. Previous stem cell treatment, autologous bone marrow transplantation or allogenic bone marrow transplantation.
10. A known history of tuberculosis.
11. Acute infection two weeks prior to baseline visit.
12. Major trauma or surgery two weeks prior to baseline.
13. Use of inhibitors of CYP3A4 within 2 weeks prior to baseline visit (1 month for fluoxetine).
14. Use of amiodarone within 2 years prior to screening visit.
15. Pregnancy or breastfeeding.
16. A ≧3×ULN serum elevation of either ALT or AST at screening.
17. Serum direct bilirubin which is 22×ULN at screening.
18. A QTc interval which is 450 msec (according to machine output) obtained from:
   a. Two ECG recordings at screening visit, or
   b. The mean value calculated from 3 baseline ECG recordings.
19. Subjects with clinically significant or unstable medical or surgical condition that would preclude safe and complete study participation, as determined by medical history, physical examination, ECG, laboratory tests or chest X-ray. Such conditions may include:
   a. A cardiovascular or pulmonary disorder that cannot be well-controlled by standard treatment permitted by the study protocol.
   b. A gastrointestinal disorder that may affect the absorption of study medication.
   c. Renal or metabolic diseases.
   d. Any form of chronic liver disease.
   e. Known human immunodeficiency virus (HIV positive status.
   f. A family history of Long-QT syndrome.
   g. A history of drug and/or alcohol abuse.
   h. Major psychiatric disorder.
20. A known history of sensitivity to Gd.
21. Inability to successfully undergo MRI scanning.
22. Known drug hypersensitivity that would preclude administration of laquinimod, such as hypersensitivity to: mannitol, meglumine or sodium stearyl fumarate.

### Outcome Measures

Neurological evaluations, including safety assessments, were performed at screening, baseline and every three months up to month 24. Patient neurological assessments and general medical evaluations were conducted by two neurologists in order to minimize the possibility of unblinding; a specially trained and certified examining neurologist assessed neurological condition, and the treating neurologist determined whether a subject had experienced a relapse based on EDSS/Functional Systems scores.

The primary endpoint was the number of confirmed relapses during the double-blind study period. A relapse was defined as the appearance of one or more new neurological abnormalities or the reappearance of one or more previously observed neurological abnormalities lasting for at least 48 hours and after an improved neurological state for at least 30 days. An event was counted as a relapse if the subject's symptoms were accompanied by observed objective neurological changes consistent with at least one of the following: an increase of at least 0.5 in the EDSS score; an increase of one grade in two or more of the seven functional systems; or an increase of two grades in one functional system. Standardized treatment of relapses was intravenous methylprednisolone 1 g/day for up to five consecutive days based on the treating neurologist's decision.

Secondary endpoints were disability progression as measured by the EDSS and the MSFC. Confirmed disability progression was defined as an increase of ≧1.0 EDSS point from baseline if baseline EDSS was between 0 and 5.0, or an increase of ≧0.5 point if baseline EDSS was ≧5.5. In order to confirm EDSS progression, these increases had to be sustained for at least three months. Additional predefined disability endpoints include the proportion of patients without confirmed disability progression at 24 months; confirmed disability progression (defined as change in EDSS scores ≧1.0 points for baseline EDSS 0 to 5.0 or ≧5.5) sustained for six months; the accumulation of physical disability as measured by mean EDSS and the mean change in EDSS from baseline to last observed value (LOV).

For the MSFC, the measure was the total MSFC z score at 24 months (including patients who terminated after 12 months). The 9-hole peg test (9HPT) and the Paced Auditory Serial Addition Test (PASAT) were performed three times at screening to reduce confounding training effects during the trial.

MRI related secondary endpoints were the cumulative number of GdE lesions at months 12 and 24; and the cumulative number of new T2 lesions (relative to previous scan) at months 12 and 24; MRI exploratory endpoints included percent change of brain volume using SIENA.¹⁰

Additional MRI methodological details are as follows: In all patients, MRI scans were performed at 0, 12, and 24 months. Before a site could enroll study participants they were required to image a volunteer patient with definite MS twice with repositioning according to a strict study imaging protocol using scanners with a minimum field strength of 1.5 T. Fast/turbo spin echo (repetition time [TR]=2200-3500 ms, echo time [TE]=14-50/90-120 ms, echo train length=2-7, slice thickness=3 mm, and contiguous axial slices=44) sequences were used to obtain proton density and T2-weighted images. High resolution pre-contrast 3D T1-weighted sequences (TR=8-15 ms, TE=3-5 ms, inversion time=1.1 s, number of slices 160, slice thickness 1.2 mm, flip angle [FA]=10-15, orientation sagittal) were acquired for quantification of brain atrophy. Finally, T1-weighted images (1.5 T scanners: conventional spin echo sequence; TR=600-650 ms, TE=10-20 ms, slice thickness=3 mm, and contiguous axial slices=44; 3.0 T scanners: 3D sequence; TR=5-9 ms, TE=2-5 ms, FA=15, slice thickness=3 mm, and contiguous axial slices=44) were obtained 5 minutes after injection of 0.1 mmol/kg of gadolinium. A series of axial, coronal, and sagittal images was obtained to create an axial reference scan for subsequent careful repositioning of each patient at the follow-up session. Axial slices were positioned to run parallel to a line joining the most inferioanterior and inferioposterior parts of the corpus callosum.

Image quality was reviewed at the MRI-AC using predetermined criteria. The identification of GdE and T2-hyperintense lesions was done by consensus of two experienced observers. The number of total and new GdE lesions and new/enlarging T2-hyperintense lesions were counted. The identified lesions were then outlined by trained technicians using a semiautomated segmentation technique based on local thresholding (Jim 4.0; Xinapse System, Leicester, UK) and lesion volumes were calculated automatically. Percentage brain volume changes and cross-sectional normalized brain volumes were measured on T1-weighted images, with Structural Image Evaluation of Normalized Atrophy (SIENA) software and a cross-sectional method (SIENAX) (available from the FMRIB Software Library, Oxford University, Oxford, UK; http://www.fmrib.ox.ac.uk/analysis/research/siena/siena).

### Results

The results of the ALLEGRO trial indicated that laquinimod treatment effectively reduced annualized relapse rates, slowed the progression of disability, reduced brain atrophy, and reduced the development of new lesions. The detailed results from ALLEGRO is disclosed in, e.g., U.S. Application Publication No. 2012-0142730, which is hereby incorporated by reference in its entirety into this application.

### Example 2

### ALLEGRO Sub-Studies

A number of ALLEGRO sub-studies were conducted to further investigate the potential neuroprotective effects of laquinimod shown in the ALLERO trial using multiple MRI techniques sensitive to irreversible tissue damage in white matter (WM) and grey matter (GM).

### Methods

### WM, GM, and Thalamic Volume Analysis.

WM, GM, and thalamic volumes were derived from 3D T1-weighted images at baseline and at months 12 and 24. Patients with baseline and at least one valid scheduled post-baseline MRI were included in the analysis. Patients with thalamic lesions at baseline and a valid post-baseline MRI were included in the thalamic lesion analysis.

Evolution of Gadolinium-Enhancing (GdE) and New T2 Lesions into Permanent Black Holes (PBH).

A subset of patients in ALLEGRO comprised a “frequent MRI” group for PBH analysis; these patients had MRIs taken at months 3, 6, 12 and 24. Patients in the frequent MRI group with active lesions at baseline or during the study were included in the PBH analysis.

### MT MRI to Determine the MT Ratio (MTR) of WM, GM, Normal Appearing Brain Tissue (NABT) and T2 Lesions.

Patients with valid baseline and month 12, and/or month 24 MT MRI assessments were included in the analysis.

### ¹H-MRS to Evaluate N-Acetylaspartate to Creatine (NAA/Cr) Ratios in WM.

Patients with baseline and month 24 or study exit ¹H-MRS assessments were included in the analysis.

### Statistical Analysis.

Efficacy analysis included all patients with at least one valid post-baseline MRI scan.

Longitudinal WM and GM volume changes were calculated as the percentage difference from baseline to months 12 and 24. A separate calculation was performed between months 12 and 24. Since these measures did not follow normal distributional assumptions, the rank values of these outcomes were analyzed as follows: a) percentage change from baseline to month 12 and 24 using a mixed model repeated measures (MMRM) (SAS® PROC MIXED) adjusted to baseline value of the outcome, number of GdE lesions at baseline, country/geographic region, visit, treatment and visit by treatment interaction; b) for percentage changes between months 12 and 24 using a separate ANCOVA model, since the MMRM model uses the baseline denominator (for % change) and thus % change from month 12 to 24 could not be derived. The ANCOVA (SAS® PROC GLM) model used treatment group, baseline value of the outcome, number of GdE lesions at baseline and country/geographic region, as covariates. Additionally, analyses of percentage changes in WM volume were also adjusted for GM volume at baseline due to the imbalance that was found between treatment groups at baseline. The Hodges-Lehmann (HL) large sample median estimator associated with the ranked values analysis approach was used to construct two-sided 95% confidence limits for the difference in treatment effects.

Percent thalamic volume changes from baseline to months 12 and 24 followed normal distribution and thus were analysed using a parametric MMRM (SAS® PROC MIXED) model corrected for baseline thalamic volume, number of baseline GdE lesions, country/geographic region, visit, treatment and visit by treatment interaction. The least square (LS) mean difference was used to estimate treatment effect. As noted for the % change in WM and GM volumes, percent thalamic volume change from month 12 to month 24 was analyzed using a separate analysis of covariance (ANCOVA) (SAS® PROC GLM) model, since these values could be derived from the MMRM model. The ANCOVA model used treatment group, baseline thalamic volume, number of baseline GdE lesions and country/geographic region, as covariates. GM at baseline was used as an additional covariate to account for between treatment group imbalance in this measure at baseline.

A negative binomial regression analysis was used to estimate the mean number of PBH at months 12 and 24 that evolved from GdE and new T2 lesions detected at various time points in the study in the “frequent MRI” subgroup.

Least square (LS) mean changes from baseline MTR in NABT, WM, GM and T2-lesions at months 12 and 24, and between months 12 an 24, were evaluated using a MMRM analysis.

Changes in NAA/Cr from baseline to month 24 was analyzed using ANCOVA.

Outcomes were assessed using all evaluable scans and all endpoints were analyzed at a significance level of 5%.

### Results

Baseline MRI measures (shown in tables 1 and 2 below) were comparable between laquinimod and placebo arms for all analysis.

TABLE 1

| Baseline measures | Laquinimod | Placebo |
|---|---|---|
| White matter fraction, | 0.32 (0.04) | 0.33 (0.04) |
| mean (SD) | (n = 478) | (n = 461) |
| | p = 0.3741* | |
| Grey matter fraction, | 0.40 (0.04) | 0.41 (0.04) |
| mean (SD) | (n = 478) | (n = 461) |
| | p = 0.0441 | |
| Thalamic fraction,^{†} | 0.0097 (0.0014) | 0.0098 (0.0015) |
| mean (SD) | (n = 476) | (n = 458) |
| | p = 0.2152 | |
| Thalamic T2 lesions volume, | 0.048 (0.1) | 0.039 (0.08) |
| ml, mean (SD) | (n = 476) | (n = 458) |

p = 0.3383
*2 patients in the laquinimod arm and 3 patients in the placebo arm with missing baseline and/or post-baseline thalamic volume data were excluded from analysis.

TABLE 2

| | PBH Sub-study | | MT MRI Sub-study | | ¹H-MRS Sub-study | |
|---|---|---|---|---|---|---|
| | Laquinimod | Placebo | Laquinimod | Placebo | Laquinimod | Placebo |
| | N = 55 | N = 69 | N = 38 | N = 37 | N = 12 | N = 15 |
| GdE lesion number | 2.6 (5.8) | 1.8 (3.0) | 0.8 (1.6) | 2.2 (4.3) | 0.6 (0.9) | 0.6 (1.1) |
| mean (SD) | p = 0.480 | | p = 0.059 | | p = 0.914 | |
| T1 hypointense lesions | 1.98 (2.38) | 1.55 (1.85) | 3.45 (6.72) | 2.61 (3.78) | 1.51 (2.51) | 0.71 (0.61) |
| volume, ml, mean (SD) | p = 0.466 | | p = 0.641 | | p = 0.782 | |
| T2 lesions volume, ml, | 8.95 (7.50) | 7.71 (7.52) | 9.81 | 9.74 (9.20) | 7.60 | 4.53 (3.04) |
| mean (SD) | | | (12.17) | | (10.21) | |
| | p = 0.237 | | p = 0.317 | | p = 0.890 | |
| Normal-appearing brain | NA | NA | 36.64 | 35.72 | NA | NA |
| tissue MTR, mean (SD) | | | (9.65) | (9.16) | | |
| | — | | p = 0.731 | | — | |
| White matter MTR, mean | NA | NA | 38.79 | 37.95 | NA | NA |
| (SD) | | | (9.64) | (9.24) | | |
| | — | | p = 0.618 | | — | |
| Grey matter MTR, mean | NA | NA | 34.64 | 33.58 | NA | NA |
| (SD) | | | (9.47) | (8.94) | | |
| | — | | p = 0.574 | | — | |
| Average MTR of T2 | NA | NA | 33.74 | 32.35 | NA | NA |
| Lesions, mean (SD) | | | (9.39) | (8.39) | | |
| | — | | p = 0.472 | | — | |
| NAA/Cr, mean (SD) | NA | NA | NA | NA | 2.79 (0.34) | 3.00 (0.44) |

| | |
|---|---|
| — | p = 0.213 |

### MRI Outcomes.

WM, GM and thalamic atrophy results are shown in table 3 below:

TABLE 3

| | White matter | Grey matter | Thalamic |
|---|---|---|---|
| | fraction | fraction | fraction |
| | median (range) | median (range) | LS mean (SE) |
| % Change from baseline to month | | | |
| 12 | | | |
| Placebo (n = 457) | −0.4 (−22.2 to 40.8) | −0.8 (−25.3 to 21.1) | −1.0 (0.12)^{‡} |
| Laquinimod (n = 472) | 0.0 (−24.7 to 11.9) | −0.3 (−19.4 to 22.4) | −0.6 (0.12) |
| p value | p = 0.004 | p = 0.004 | p = 0.005 |
| Treatment effect | 0.394* | 0.502* | 0.408^{†} |
| % Change from month 12 to | | | |
| month 24 | | | |
| Placebo (n = 409) | −0.2 (−21.1 to 10.6) | −0.6 (−14.3 to 32.5) | −0.9 (0.13)^{§} |
| Laquinimod (n = 419) | −0.2 (−15.3 to 15.0) | −0.7 (−11.5 to 16.6) | −0.7 (0.13) |
| p value | p = 0.857 | p = 0.664 | p = 0.13 |
| Treatment effect | −0.013* | −0.078* | 0.233^{†} |
| % Change from baseline to month | | | |
| 24 | | | |
| Placebo (n = 409) | −0.5 (−25.7 to 39.8) | −1.2 (−16.9 to 24.3) | −1.8 (0.15)^{\|\|} |
| Laquinimod (n = 418) | −0.3 (−27.3 to 19.2) | −0.9 (−17.4 to 21.2) | −1.3 (0.15) |
| p value | p = 0.035 | p = 0.078 | p = 0.003 |
| Treatment effect | 0.327* | 0.372* | 0.60^{†} |

*Hodges-Lehmann median estimate for treatment difference.
^{†}LS mean difference.
^{‡}Evaluable for thalamic volume at month 12: n = 468 laquinimod, n = 454 placebo.
^{§}Evaluable for thalamic volume at both month 12 and month 24: n = 402 laquinimod, n = 400 placebo.
^{||}Evaluable for thalamic volume at month 24: n = 408 laquinimod, n = 404 placebo.

Patients treated with laquinimod showed lower percentage losses of WM and GM volume at month 12 (p=0.004 for both) and differences at month 24 were statistically significant for WM, while approached statistical significance for GM (p=0.035 and p=0.078). In addition, percent reduction from baseline thalamic volume was significantly lower in laquinimod-treated patients at months 12 (p=0.005) and 24 (p=0.003) compared with placebo. Moreover, for patients with thalamic lesions at baseline, the treatment effect of laquinimod on thalamic fraction was statistically significant at 12 (p=0.018) and 24 months (p<0.0001), with a significant correlation between change in T2 thalamic lesion volume and change in thalamic fraction from baseline to month 24 (r=−0.184, p<0.001). However, for patients without T2 thalamic lesions at baseline, the treatment effect of laquinimod on thalamic fraction change was not significantly different from that of placebo at month 12 or 24.

PB results are shown in table 4 below:

TABLE 4

| | GdE lesions | | | New T2 lesions | | | Active lesions | | |
|---|---|---|---|---|---|---|---|---|---|
| | Mean* (SE) | | | Mean (SE) | | | Mean* (SE) | | |
| | [n patients | | | [n patients | | | [n patients | | |
| | evaluable] | RR^{†} | P value | evaluable] | RR^{†} | P value | evaluable] | RR^{†} | P value |
| PBH at Month 12 from | | | | | | | | | |
| lesions at Baseline | | | | | | | | | |
| Laquinimod 0.6 mg | 0.5 (0.16) [26] | 0.53 | 0.112 | NA | NA | NA | NA | NA | NA |
| Placebo | 0.9 (0.19) [41] | | | | | | | | |
| PBH at Month 24 from | | | | | | | | | |
| lesions at Baseline | | | | | | | | | |
| Laquinimod 0.6 mg | 0.6 (0.17) [26] | 0.65 | 0.184 | NA | NA | NA | NA | NA | NA |
| Placebo | 1.0 (0.17) [41] | | | | | | | | |
| PBH at Month 12 from | | | | | | | | | |
| lesions at Month 3 | | | | | | | | | |
| Laquinimod 0.6 mg | 0.6 (0.25) [14] | 0.58 | 0.244 | 0.40 (0.15) [28] | 0.52 | 0.143 | 0.41 (0.14) [29] | 0.44 | 0.040 |
| Placebo | 1.0 (0.22) [37] | | | 0.77 (0.20) [42] | | | 0.94 (0.20) [45] | | |
| PBH at Month 24 from | | | | | | | | | |
| lesions at Month 3 | | | | | | | | | |
| Laquinimod 0.6 mg | 0.6 (0.24) [14] | 0.55 | 0.193 | 0.43 (0.14) [28] | 0.50 | 0.076 | 0.47 (0.14) [29] | 0.45 | 0.025 |
| Placebo | 1.1 (0.22) [37] | | | 0.86 (0.18) [42] | | | 1.03 (0.19) [45] | | |
| PBH at Month 12 from | | | | | | | | | |
| lesions at Months 3 and 6 | | | | | | | | | |
| Laquinimod 0.6 mg | 0.6 (0.18) [25] | 0.45 | 0.022 | 0.57 (0.16) [40] | 0.48 | 0.030 | 0.59 (0.15) [41] | 0.42 | 0.004 |
| Placebo | 1.3 (0.22) [50] | | | 1.20 (0.24) [56] | | | 1.40 (0.23) [58] | | |
| PBH at Month 24 from | | | | | | | | | |
| lesions at Months 3, 6, and | | | | | | | | | |
| 12 | | | | | | | | | |
| Laquinimod 0.6 mg | 0.7 (0.19) [32] | 0.44 | 0.005 | 0.87 (0.17) [51] | 0.52 | 0.009 | 0.95 (0.18) [52] | 0.50 | 0.002 |
| Placebo | 1.7 (0.26) [54] | | | 1.67 (0.25) [65] | | | 1.91 (0.26) [66] | | |
| PBH at Month 24 from All | | | | | | | | | |
| lesions | | | | | | | | | |
| Laquinimod 0.6 mg | 1.0 (0.20) [41] | 0.45 | 0.001 | 0.87 (0.17) [51] | 0.52 | 0.009 | 1.20 (0.19) [55] | 0.51 | <0.001 |
| Placebo | 2.1 (0.29) [60] | | | 1.67 (0.25) [65] | | | 2.34 (0.28) [69] | | |

*Adjusted means;
^{†}Laquinimod vs placebo;
NA = not assessed, since “new” T2 lesions were not available at baseline.
Data from a negative binomial regression model.
PBH = permanent black hole.
GdE = gadolinium enhancing
RR = risk ratio.

The mean number of PBH that developed from only GdE lesions detected while on treatment was lower with laquinimod vs placebo at month 12 (from GdE lesions detected at months 3 and 6, risk ratio=0.45, p=0.022) and at month 24 (from GdE lesions at months 3, 6, and 12, risk ratio=0.44, p=0.005). A similar trend was apparent for the mean numbers of PBH at months 12 and 24 that evolved from new T2 lesions in laquinimod vs placebo comparisons (p=0.030 and p=0.009, respectively). There was no difference between placebo and laquinimod at 12 and 24 months in the number of PBH evolved from GdE lesions present at baseline. However, if all GdE lesions were included in the analysis (those detected at baseline and while on treatment), the mean number of PBH at month 24 was lower with laquinimod vs placebo (risk ratio=0.45, p=0.001).

MT MRI results are shown in table 5 below:

| | Laquinimod 0.6 mg | Placebo (N = 37) |
|---|---|---|
| | (N = 38) | LS mean |
| | LS mean (SE) change | (SE) change |
| NABT | | |
| Baseline to Month 12 | (n = 38) | (n = 37) |
| | 0.31 (0.12) | −0.09 (0.12) |
| Treatment difference | 0.40 (0.09, 0.71) p = 0.013 | |
| (95% CI) | | |
| Month 12 to Month 24 | (n = 31) | (n = 32) |
| | −0.08 (0.14) | −0.18 (0.14) |
| Treatment difference | 0.10 (−0.31, 0.50) p = 0.642 | |
| Baseline to Month 24 | (n = 31) | (n = 32) |
| | 0.23 (0.15) | −0.27 (0.14) |
| Treatment difference | 0.50 (0.10, 0.89) p = 0.015 | |
| WM | | |
| Baseline to Month 12 | (n = 38) | (n = 37) |
| | 0.32 (0.13) | −0.09 (0.12) |
| Treatment difference | 0.40 (0.09, 0.72) p = 0.013 | |
| Month 12 to Month 24 | (n = 31) | (n = 32) |
| | −0.05 (0.13) | −0.18 (0.13) |
| Treatment difference | 0.13 (−0.24, 0.51) p = 0.486 | |
| Baseline to Month 24 | (n = 31) | (n = 32) |
| | 0.27 (0.16) | −0.27 (0.15) |
| Treatment difference | 0.53 (0.13, 0.94) p = 0.011 | |
| GM | | |
| Baseline to Month 12 | (n = 38) | (n = 37) |
| | 0.30 (0.13) | −0.11 (0.12) |
| Treatment difference | 0.41 (0.08, 0.74) p = 0.014 | |
| Month 12 to Month 24 | (n = 31) | (n = 32) |
| | −0.16 (0.15) | −0.22 (0.15) |
| Treatment difference | 0.06 (−0.37, 0.49) p = 0.787 | |
| Baseline to Month 24 | (n = 31) | (n = 32) |
| | 0.14 (0.16) | −0.33 (0.16) |
| Treatment difference | 0.47 (0.04, 0.90) p = 0.034 | |
| Average MTR of T2 lesions | | |
| Baseline to Month 12 | (n = 37) | (n = 37) |
| | 0.39 (0.25) | 0.02 (0.24) |
| Treatment difference | 0.38 (−0.26, 1.01) p = 0.239 | |
| Month 12 to Month 24 | (n = 30) | (n = 32) |
| | 0.07 (0.24) | −0.08 (0.23) |
| Treatment difference | 0.15 (−0.51, 0.81) p = 0.651 | |
| Baseline to Month 24 | (n = 30) | (n = 32) |
| | 0.46 (0.29) | −0.07 (0.28) |

| | |
|---|---|
| Treatment difference | 0.53 (−0.23, 1.28) p = 0.168 |

NABT = normal appearing brain tissue;
WM = white matter;
GM = grey matter;
LS = least square;
MTR = magnetization transfer ratio;
CI = confidence intervals.

LS mean changes in MTR values with placebo decreased from baseline to months 12 and 24 and between months 12 and 24 for all assessments, MTR values in the laquinimod group increased from baseline to months 12 and 24 for all MTR outcomes and slightly decreased from month 12 to month 24 for all assessments except for T2 lesion, which slightly increased.

Significant differences between treatment arms in LS mean change in MTR values favored laquinimod at months 12 and 24 for NABT (p=0.013 and p=0.015, respectively), WM (p=0.013 and p=0.011), and GM (p=0.014 and p=0.034). Changes in LS mean MTR of T2 lesions did not differ significantly between laquinimod and placebo patients at any time point. No significant treatment differences were found for any MTR measures between month 12 and month 24.

### ¹H MRS Results:

At 24 months, WM NAA/Cr ratio tended to increase with laquinimod and decrease with placebo though the difference was not statistically significant (p=0.179) in this small patient population (N=27)

### CONCLUSIONS

Laquinimod reduced brain volume loss in both WM and GM compared with placebo, with more pronounced effects in the first year of treatment.

Studies suggest thalamic atrophy may be more clinically relevant than volume loss in the entire GM (Rocca 2010 and Audoin 2006). Laquinimod significantly reduced thalamic atrophy at 12 and 24 months compared with placebo. Treatment effect was most apparent in patients with thalamic lesions before beginning laquinimod treatment.

Taken together, these findings, from a variety of MRI techniques, suggest a neuroprotective effect of oral laquinimod in patients with RRMS, and are consistent with clinical benefits of laquinimod shown in the phase III ALLEGRO and BRAVO studies (Comi 2012 and Vollmer 2011).

### Example 3

### Assessment of Oral Laquinimod in Treating Tremor and Spasticity

It has been suggested that spasticity or tremor can be caused by damages to the thalamus, and stimulation of the thalamus can be beneficial for treating tremor and spasticity.

A composition comprising laquinimod as described herein is administered to a subject afflicted by tremor. The administration of the composition is effective to inhibit tremor in the subject.

A composition comprising laquinimod as described herein is administered to a subject afflicted by tremor. The administration of the composition is effective to reduce tremor in the subject.

A composition comprising laquinimod as described herein is administered to a subject afflicted by spasticity. The administration of the composition is effective to inhibit spasticity in the subject.

A composition comprising laquinimod as described herein is administered to a subject afflicted by tremor. The administration of the composition is effective to reduce spasticity in the subject.

### REFERENCES

1. Audoin et al. Localization of grey matter atrophy in early RRMS: A longitudinal study. J Neurol 2006; 253: 1495-501.
2. Barkhof, F. (1999) “MRI in Multiple Sclerosis: Correlation with Expanded Disability Status Scale (EDSS)”, Multiple Sclerosis. 5(4):283-286 (Abstract).
3. Bar-Zohar et al. Magnetic resonance imaging metrics and their correlation with clinical outcomes in multiple sclerosis: a review of the literature and future perspectives. Mult Scler 2008; 14: 719-27.
4. Bermel et al. The measurement and clinical relevance of brain atrophy in multiple sclerosis. Lancet Neurol 2006; 5: 158-70.
5. Bjartmar and Fox (2002) “Pathological mechanisms and disease progression of multiple sclerosis: therapeutic implication”, Drugs of Today. 38:7-29.
6. Brex et al. (2002) “A longitudinal study of abnormalities on MRI and disability from multiple sclerosis”, N Engl J Med. Jan. 17, 2002 346(3):158-64.
7. Brod et al. (2000) Annals of Neurology, 47:127-131.
8. Brück (2011) “Insight into the mechanism of laquinimod action.” J Neurol Sci. 2011 Jul. 15; 306 (1-2):173-9.
9. Bruck et al. Reduced astrocytic NF-kappaB activation by laquinimod protects from cuprizone-induced demyelination. Acta Neuropathol 2012; 124: 411-24.
10. Brunmark et al. (2002) “The new orally active immunoregulator laquinimod (ABR-215062) effectively inhibits development and relapses of experimental autoimmune encephalomyelitis”, J Neuroimmunology. 130:163-172.
11. Cambier J. (1982) “[Dejerine-Roussy syndrome]” Rev Neurol (Paris) 138(12):979-88 (Abstract, article in French).
12. Cohen et al. (2010) “Oral fingolimod or intramuscular interferon for relapsing multiple sclerosis”. N Eng J Med; 362:402-415.
13. Comi et al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 59th Annual Meeting of the American Academy of Neurology; April 28-May 5, 2007; Boston, Mass.
14. Comi et al. (2008) “Effect of laquinimod on MRI-monitored disease activity in patients with relapsing-remitting multiple sclerosis: a multicentre, randomised, double-blind, placebo-controlled phase IIb study”, Lancet. 371:2085-2092.
15. Comi et al. (2009) for the LAQ/5062 Clinical Advisory Board and Study Group. Long-term open extension of oral laquinimod in patients with relapsing multiple sclerosis shows favorable safety and sustained low relapse rate and MRI activity. [Ectrims abstract P443]. Mult Scier. 15 (Suppl 2):S127.
16. Comi et al. (2010) for the LAQ/5062 Clinical Advisory Board and Study Group. The effect of laquinimod on MRI-monitored disease activity in patients with relapsing-remitting multiple sclerosis: a double-blind active extension of the multicentre, randomised, double-blind, parallel-group placebo-controlled study. Mult Scler. 16:1360-1366.
17. Comi et al. Placebo-controlled trial of oral laquinimod for multiple sclerosis. New Engl J Med 2012; 366:1000-9.
18. Cutter et al. (1999) “Development of a multiple sclerosis functional composite as a clinical trial outcome measure”, Brain. 122:871-882.
19. De Stefano et al. (1999) “Evidence of early axonal damage in patients with multiple sclerosis”, Neurology. 52 (Suppl 2):A378.
20. Dunitz. M. (1999) Multiple sclerosis therapeutics, Ed. Rudick and Goodkin. London: Taylor & Francis, 1999.
21. Durelli et al. and the Independent Comparison of Interferon (INCOMIN) Trial Study Group. (2002) “Every-other-day interferon beta-1b versus once-weekly interferon beta-1a for multiple sclerosis: results of a 2-year prospective randomised multicentre study (INCOMIN)”, Lancet. 359:1453-60.
22. EMEA Guideline on Clinical Investigation of Medicinal Products for the Treatment of Multiple Sclerosis (CPMP/EWP/561/98 Rev. 1, Nove. 2006).
23. EPAR, Rebif®, Scientific Discussion.
24. FDA 2005. Draft Guidance for Industry—Systemic Lupus Erythematosus—Developing Drugs for Treatment (http://www.fda.gov/downloads/Drugs/GuidanceComplianceRegula toryInformation/Guidances/ucm072063.pdf).
25. Filippi et al., Pridopidine reduces the proportion of MS lesions evolving into black holes, Neurol., 2001, 57:731-733.
26. Fischer et al. (1999) “The Multiple Sclerosis Functional Composite measure (MSFC): an integrated approach to MS clinical outcome assessment” Multiple Sclerosis. 5(4):244-250.
27. Fisk et al. (1994) “Measuring the Functional Impact of Fatigue: Initial Validation of Fatigue Impact Scale”, Clin Inf Dis. 18 Suppl 1:S79-83.
28. Fisk et al. (1994) “The Impact of Fatigue on Patients with Multiple Sclerosis”, Can J Neurol Sci. 21:9-14.
29. Frohman et al. (2003) “The utility of MRI in suspected MS: report of the Therapeutics and Technology Assessment Subcommittee of the American Academy of Neurology”, Neurology. Sep. 9, 2003, 61(5):602-11.
30. Giovannoni et al. (2010) “A placebo-controlled trial of oral cladribine for relapsing multiple sclerosis”, N Eng J Med. 362:416-426.
31. Golder W. (2007) “Magnetic resonance spectroscopy in clinical oncology”, Onkologie. 27(3): 304-9.
32. Grossman et al. (1994) Magnetization transfer: theory and clinical applications in neuroradiology”, RadioGraphics. 14:279-290.
33. Gurevich et al. (2010) “Laquinimod suppress antigen presentation in relapsing-remitting multiple sclerosis: in-vitro high-throughput gene expression study” (J Neuroimmunol. 2010 Apr. 15; 221 (1-2):87-94. Epub 2010 March 27.
34. Hartung et al. (2005) “Significance of neutralizing antibodies to interferon beta during treatment of multiple sclerosis: expert opinions based on the Proceedings of an International Consensus Conference”, Eur J Neurol. 12:588-601.
35. Hohlfeld et al. (2000) “The neuroprotective effect of inflammation: implications for the therapy of multiple sclerosis”, J Neuroimmunol. 107:161-166.
36. Jacobs et al. (1996) “Intramuscular interferon beta-1a for disease progression in relapsing multiple sclerosis”, Ann Neurol. 39:285-294.
37. Jong et al. (2008) “Strongly reduced volumes of putamen and thalamus in Alzheimer's disease: an MRI study” Brain (2008), 131:3277-3285.
38. Kappos et al. (2010) “A placebo-controlled trial of oral fingolimod in relapsing multiple sclerosis”, N Eng J Med. 362:387-401.
39. Kassubet et al. (2005) “Thalamic Atrophy in Huntington's Disease Co-varies with Cognitive Performance: A Morphometric MRI Analysis” Cereb. Cortex (June 2005) 15(6):846-853.
40. Kim (2001) “Delayed onset mixed involuntary movements after thalamic stroke, clinical, radiological and pathophysiological findings” Brain (2001), 124:299-309.
41. Kleinschmidt-DeMasters et al. (2005) New England Journal of Medicine, 353:369-379.
42. Kurtzke J F. (1983) “Rating neurologic impairment in multiple sclerosis: an expanded disability status scale (EDSS)”, Neurology 33(11):1444-1452.
43. Langer-Gould et al. (2005) New England Journal of Medicine, 353:369-379.
44. Lee and Marsden (1994) “Movement disorders following lesions of the thalamus or subthalamic region” Mov Disord. 9 (5); 493-507.
45. Lublin and Reingold (1996) “Defining the clinical course of multiple sclerosis”, Neurol. 46:907-911.
46. McDonald, (2001) “Guidelines from the International Panel on the Diagnosis of Multiple Sclerosis” Ann. Neurol. 50:121-127.
47. Mehta et al. (1996) “Magnetization transfer magnetic resonance imaging: a clinical review”, Topics in Magnetic Resonance Imaging 8(4):214-30.
48. Miki et al. (1999) “Relapsing-Remitting Multiple Sclerosis: Longitudinal Analysis of MR Images—Lack of Correlation between Changes in T2 Lesion Volume and Clinical Findings”, Radiology. 213:395-399.
49. Miller et al. (2007) “MRI outcomes in a placebo-controlled trial of natalizumab in relapsing MS”, Neurology. 68:1390-1401.
50. Neuhaus et al. (2003) “Immunomodulation in multiple sclerosis: from immunosuppression to neuroprotection”, Trends Pharmacol Sci. 24:131-138.
51. Noseworthy et al. (2000) “Multiple sclerosis”, N Engl J Med. 343:938-952.
52. Noseworthy et al. (2000) “Linomide in relapsing and secondary progressive MS. Part 1: Trial Design and clinical results”, Neurology. 54:1726-1733.
53. Panitch H, Goodin D S, Francis G, Chang P, Coyle P K, O'Connor P, Monaghan E, Li D, Weinsjenker B, for the EVIDENCE (Evidence of Interferon Dose-response: European North American Comparative Efficacy) Study Group and the University of British Columbia MS/MRI Research Group. (2002) “Randomized comparative study of interferon β-1a treatment regiments in MS”, The EVIDENCE Trial. Neurology. 59:1496-1506.
54. PCT International Application Publication No. WO 1998/30227, published Jul. 16, 1998.
55. PCT International Application Publication No. WO 2000/05250, published Feb. 3, 2000.
56. PCT International Application Publication No. WO 2000/18794, published Apr. 6, 2000.
57. PCT International Application Publication No. WO 2001/46145, published Jun. 28, 2001.
58. PCT International Application Publication No. WO 2003/048735, published Jun. 12, 2003.
59. PCT International Application Publication No. WO 2004/103297, published Dec. 2, 2004.
60. PCT International Application Publication No. WO 2006/016036, published Nov. 2, 2006.
61. PCT International Application Publication No. WO 2006/029393, published Mar. 16, 2006.
62. PCT International Application Publication No. WO 2006/029411, published Mar. 16, 2006.
63. PCT International Application Publication No. WO 2006/040155, published Apr. 20, 2006.
64. PCT International Application Publication No. WO 2006/083608, published Aug. 10, 2006.
65. PCT International Application Publication No. WO 2006/089164, published Aug. 24, 2006.
66. PCT International Application Publication No. WO 2006/116602, published Nov. 2, 2006.
67. PCT International Application Publication No. WO 2007/047863, published Apr. 26, 2007, international filing date Oct. 18, 2006.
68. PCT International Application Publication No. WO 2007/146248, published Dec. 21, 2007.
69. PCT International Application Publication No. WO 2007/146248, published Dec. 21, 2007, international filing date Jun. 12, 2007.
70. PCT International Application Publication No. WO 2009/070298, published Jun. 4, 2009.
71. PCT International Application Publication No. WO 2011/008274, published Jan. 20, 2011.
72. PCT International Application Publication No. WO 2011/022063, published Feb. 24, 2011.
73. PCT International Application Publication No. WO 2011/107583, published Sep. 9, 2011.
74. PCT International Application Publication No. WO 2012/051106, published Apr. 19, 2012.
75. Polman et al. (2005) “Diagnostic criteria for multiple sclerosis: 2005 revisions to the McDonald Criteria”, Annals of Neurology, Volume 58 Issue 6, Pages 840-846.
76. Polman et al. (2005) “Treatment with laquinimod reduces development of active MRI lesions in relapsing MS”, Neurology. 64:987-991.
77. Polman et al. (2006) “A randomized, placebo-controlled trial of natalizumab for relapsing multiple sclerosis”, N Eng J Med. 354:899-910.
78. Poser et al. (1983) “New Diagnostic Criteria for Multiple Sclerosis: Guidelines for Research Protocols”, Annals of Neurology, March 1983, 13(3):227-230.
79. Preiningerova J. (2009) “Oral laquinimod therapy in relapsing multiple sclerosis”, Expert Opin Investig Drugs. 18:985-989.
80. PRISMS Study Group. Randomized double-blind placebo-controlled study of interferon β-1a in relapsing/remitting multiple sclerosis. Lancet 1998; 352:1498-1506.
81. Rocca et al. Thalamic damage and long-term progression of disability in multiple sclerosis. Radiology 2010; 257: 463-9.
82. Rosen Y. (2007) “The Recent advances in magnetic resonance neurospectroscopy”, Neurotherapeutics. 27(3): 330-45.
83. RTT News Article dated April 12, 11, entitled “Teva Pharma, Active Biotech Post Positive Laquinimod Phase 3 ALLEGRO Results”.
84. Rudick et al. (1999) “Use of the brain parenchymal fraction to measure whole brain atrophy in relapsing-remitting MS: Multiple Sclerosis Collaborative Research Group”. Neurology. 53:1698-1704.
85. Rudick et al. (2006) New England Journal of Medicine, 354:911-923.
86. Rudick, R. (1999) “Disease-Modifying Drugs for Relapsing-Remitting Multiple Sclerosis and Future Directions for Multiple Sclerosis Therapeutics”, Neurotherpatueics. 56:1079-1084.
87. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
88. Runström et al. (2006) “Inhibition of the development of chronic experimental autoimmune encephalomyelitis by laquinimod (ABR-215062) in IFN-β k.o. and wild type mice” Journal of Neuroimmunology, 173(2006):69-78.
89. Sandberg-Wollheim et al. (2005) “48-week open safety study with high-dose oral laquinimod in patients”, Mult Scler. 11:S154 (Abstract).
90. Sheline, Yvette I. (2003) “Neuroimaging Studies of Mood Disorder Effects on the Brain” Biol Psychiatry, 54:338-352.
91. SIENA and SIENAX available from the FMRIB Software Library, Oxford University. Oxford, UK;http://www.fmrib.ox.ac.uk/analysis/research/siena/siena.
92. Sorenson P S. (2006) “Neutralising antibodies to interferon-β—measurement, clinical relevance, and management”, J Neurol. 253[Suppl 6]:VI/16-VI/22.
93. Sormani et al. (2004) “Measurement error of two different techniques for brain atrophy assessment in multiple sclerosis”, Neurology. 62:1432-1434.
94. Stachnowiak, Julie (2007) “Thalamus” About.com Guide, updated May 29, 2007<http://ms.about.com/od/glossary/g/thalamus.htm>, retrieved on Oct. 12, 2012.
95. Temple R. (2006) “Hy's law: predicting serious hepatoxicity”, Pharmacoepidemiol Drug Saf. 15(4):241-3.
96. The IFNB Multiple Sclerosis Study Group. (1993) Interferon beta-1b is effective in relapsing-remitting multiple sclerosis. I. Clinical results of a multicenter, randomized, double-bind, placebo-controlled trial. Neurology; 43:655-661.
97. The IFNB Multiple Sclerosis Study Group. (1993) Interferon beta-1b is effective in relapsing-remitting multiple sclerosis. II. MRI analysis results of a multicenter, randomized, double-blind, placebo-controlled trial. Neurology; 43:662-667.
98. The National MS Society (USA), The Disease Modifying Drug Brochure, Oct. 19, 2006.
99. Thöne and Gold (2011) “Laquinimod: a promising oral medication for the treatment of relapsing-remitting multiple sclerosis”, Expert Opin Drug Metab Toxicol. 2011 March; 7(3): 365-70.
100. Torres, et al. (2010) “Deep Brain Stimulation of the Ventral Intermediate Nucleus of the Thalamus for Tremor in Patients with Multiple Sclerosis.” Neurosurgery, 67(3):646-651.
101. Trinadad Herrero, et al. (2002) “Functional anatomy of the thalamus and basal ganglia” Child's Nerv Syst (2002), 18:386-404.
102. Tuling and Tunks (1999) “Thalamic Pain Syndrome (Central Post-Stroke Pain) in a patient presenting with right upper limb pain: a case report” J Can Chiropr Assoc 43(4):243-248.
103. Turner et al. Cerebral atrophy and disability in relapsing-remitting and secondary progressive multiple sclerosis over four years. Mult Scler 2003; 9: 21-7.
104. U.S. Patent Application Publication No. 2008-0207526, published Aug. 28, 2008 (Strominger et al.).
105. U.S. Patent Application Publication No. 2010-0197712, published Aug. 5, 2010 (Carlsson et al.).
106. U.S. Patent Application Publication No. 2010-0322900, published Dec. 23, 2010 (Tarcic et al.).
107. U.S. Patent Application Publication No. 2011-0027219, published Feb. 3, 2011 (Tarcic et al.).
108. U.S. Patent Application Publication No. 2011-0034508, published Feb. 10, 2011 (Liat Hayardeny).
109. U.S. Patent Application Publication No. 2011-0206782, published August 25, 11 (Zhang).
110. U.S. Patent Application Publication No. 2031-0217295, published Sep. 8, 2011 (Haviv and Tarcic).
111. U.S. Patent Application Publication No. 2011-0218179, published Sep. 8, 2011 (Haviv and Tarcic).
112. U.S. Patent Application Publication No. 2011-0218203, published Sep. 8, 2011 (Joel Kaye et al.).
113. U.S. Patent Application Publication No. 2012-0010238, published Jan. 12, 2012 (Fristedt).
114. U.S. Patent Application Publication No. 2012-0010239, published Jan. 12, 2012 (Piryatinsky et al.).
115. U.S. Patent Application Publication No. 2012-0142730, published Jun. 7, 2012 (Tarcic et al.).
116. U.S. Pat. No. 3,849,550, issued Nov. 19, 1974 (Teitelbaum et al).
117. U.S. Pat. No. 5,800,808, issued Sep. 1, 1998 (Konfino et al).
118. U.S. Pat. No. 5,858,964, issued Jan. 12, 1999 (Aharoni et al).
119. U.S. Pat. No. 5,981,589, issued Nov. 9, 1999 (Konfino et al).
120. U.S. Pat. No. 6,048,898, issued Apr. 11, 2000 (Konfino et al).
121. U.S. Pat. No. 6,054,430, issued Apr. 25, 2000 (Konfino et al).
122. U.S. Pat. No. 6,077,851, issued Jun. 20, 2000 (Bjork et al).
123. U.S. Pat. No. 6,214,791, issued Apr. 10, 2001 (Arnon et al).
124. U.S. Pat. No. 6,342,476, issued Jan. 29, 2002 (Konfino et al).
125. U.S. Pat. No. 6,362,161, issued Mar. 26, 2002 (Konfino et al).
126. U.S. Pat. No. 7,566,767, issued Jul. 28, 2009 (Strominger et al.).
127. U.S. Pat. No. 7,589,208, issued Sep. 15, 2009 (Jansson et al).
128. U.S. Pat. No. 7,884,208, issued Feb. 8, 2011 (Frenkel et al.).
129. U.S. Pat. No. 7,989,473, issued Aug. 2, 2011 (Patashnik et al.).
130. U.S. Pat. No. 8,178,127, issued May 15, 2012 (Safadi et al.).
131. U.S. Pat. No. 8,252,993, issued Aug. 28, 2012 (Gant and Shahbaz).
132. US Food and Drug Administration, Center for Drug Evaluation and Research. Peripheral and Central Nervous System (PCNS) Advisory Committee. US Department of Health and Human Services 2006. Briefing Document. Biogen Idec Biologics Marketing Application STN 125104/15. Natalizumab (Tysabri) for Multiple Sclerosis. Dated Feb. 9, 2006. Pages 45-48.
133. Vollmer et al. A placebo-controlled and active comparator phase III trial (BRAVO) for relapsing-remitting multiple sclerosis. 5th Joint Triennial Congress of the European and Americas Committees for Treatment and Research in Multiple Sclerosis. October 19-22, 2011; Amsterdam, The Netherlands: 8.
134. Wegner et al. Laquinimod interferes with migratory capacity of T cells and reduces IL-17 levels, inflammatory demyelination and acute axonal damage in mice with experimental autoimmune encephalomyelitis. J Neuroimmunol 2010; 227:133-43.
135. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
136. Zou et al. (2002) “Suppression of experimental autoimmune neuritis by ABR-215062 is associated with altered Th1/Th2 balance and inhibited migration of inflammatory cells into the peripheral nerve tissue”, Neuropharmacology. 42:731.

What is claimed is:

1. A method for inhibiting or reducing thalamic damage in a subject afflicted with a form multiple sclerosis or presenting a clinically isolated syndrome, comprising orally administering to the subject an amount of laquinimod so as to thereby inhibit or reduce thalamic damage in the subject, wherein the subject is a human patient who has been determined to have thalamic damage at baseline.

2. The method of claim 1, wherein the form of multiple sclerosis is relapsing-remitting multiple sclerosis.

3. The method of claim 1, wherein the form of multiple sclerosis is a progressive form of multiple sclerosis.

4. The method of any one of claims 1-3, wherein the patient is a naïve patient.

5. The method of any one of claims 1-4, wherein the patient has previously received at least one multiple sclerosis therapy.

6. The method of any one of claims 1-5, wherein the subject has been determined to have at least one thalamic lesion at baseline.

7. The method of claim 6, wherein the thalamic lesion is a T2 thalamic lesion

8. A method for inhibiting or reducing thalamic damage in a subject afflicted with a disease or disorder other than a form of multiple sclerosis or a clinically isolated syndrome, comprising administering to the subject an amount of laquinimod so as to thereby inhibit or reduce thalamic damage in the subject.

9. The method of claim 8, wherein the subject is a human.

10. The method of claims 8 or 9, wherein the subject is not afflicted with a form of multiple sclerosis and is not presenting a clinically isolated syndrome.

11. The method of any one of claims 8-10, wherein the subject is a naïve subject.

12. The method of any one of claims 8-11, wherein the subject is afflicted with a movement disorder and the administration of laquinimod is effective to treat the subject.

13. The method of claim 12, wherein the movement disorder is dystonia, paroxysmal dystonia, asterixis, chorea, ataxia, ballism-chorea, myorhythmic movements, dyskinesia, bepharospasm, epilepsy, seizures or convulsions.

14. The method of any one of claims 8-13, wherein the subject is afflicted with a mood disorder and the administration of laquinimod is effective to treat the subject.

15. The method of claim 14, wherein the mood disorder is depression, anxiety or bipolar disorder.

16. The method of any one of claims 8-15, wherein the subject is afflicted with Parkinson's disease, Alzheimer's disease, schizophrenia or Huntington's disease and the administration of laquinimod is effective to treat the subject.

17. The method of any one of claims 8-16, wherein the subject is afflicted with thalamic pain syndrome and the administration of laquinimod is effective to treat the subject.

18. The method of any one of claims 8-17, wherein the subject has been determined to have thalamic damage at baseline.

19. The method of any one of claims 1-18, wherein the thalamic damage is a thalamic lesion.

20. The method of claim 19, wherein the thalamic lesion is a T2 thalamic lesion

21. The method of any one of claims 1-20, wherein the thalamic damage is measured using Magnetic resonance imaging (MRI).

22. The method of any one of claims 1-21, wherein the subject is afflicted by tremor or spasticity.

23. The method of any one of claims 1-22, where the subject is a human patient diagnosed to be afflicted by tremor or spasticity.

24. The method of claim 23, wherein the subject is diagnosed to be afflicted by tremor or spasticity which is treatable by laquinimod.

25. The method of any one of claims 1-24, wherein the administration of laquinimod is effective to reduce or inhibit tremor in the subject.

26. The method of any one of claims 1-25, wherein the administration of laquinimod is effective to reduce or inhibit spasticity in the subject.

27. The method of any one of claims 1-26, wherein the subject has previously suffered a thalamic stroke.

28. The method of any one of claims 1-27, wherein laquinimod is administered via oral administration.

29. The method of any one of claims 1-28, wherein laquinimod is administered periodically.

30. The method of claim 29, wherein the periodic administration is for a period of greater than 24 weeks.

31. The method of any one of claims 1-30, wherein laquinimod is administered daily.

32. The method of any one of claims 1-30, wherein laquinimod is administered more often than once daily.

33. The method of any one of claims 1-30, wherein laquinimod is administered less often than once daily.

34. The method of any one of claims 1-33, wherein the amount laquinimod administered is 0.1-2.5 mg/day.

35. The method of claim 34, wherein the amount laquinimod administered is 0.25-2.0 mg/day.

36. The method of claim 35 wherein the amount laquinimod administered is 0.3-0.9 mg/day.

37. The method of claim 35, wherein the amount laquinimod administered is 0.5-1.2 mg/day.

38. The method of claim 34, wherein the amount laquinimod administered is 0.25 mg/day.

39. The method of claim 34, wherein the amount laquinimod administered is 0.3 mg/day.

40. The method of claim 34, wherein the amount laquinimod administered is 0.5 mg/day.

41. The method of claim 34, wherein the amount laquinimod administered is 0.6 mg/day.

42. The method of claim 34, wherein the amount laquinimod administered is 1.0 mg/day.

43. The method of claim 34, wherein the amount laquinimod administered is 1.2 mg/day.

44. The method of claim 34, wherein the amount laquinimod administered is 1.5 mg/day.

45. The method of claim 34, wherein the amount laquinimod administered is 2.0 mg/day.

46. A method for inhibiting or reducing tremor or spasticity in a subject afflicted by tremor or spasticity, comprising administering to the subject an amount of laquinimod so as to thereby inhibit or reduce the tremor or the spasticity in the subject.

47. The method of claim 46, where the subject is a human patient afflicted with a form multiple sclerosis or presenting a clinically isolated syndrome.

48. The method of claim 46, where the subject is a human patient not afflicted with a form multiple sclerosis or presenting a clinically isolated syndrome.

49. The method of any one of claims 46-48, wherein the subject is a human patient diagnosed to be afflicted by tremor or spasticity.

50. The method of claim 49, wherein the subject is afflicted by tremor or spasticity which is treatable by laquinimod.

51. Laquinimod for use in inhibiting or reducing thalamic damage in a human patient who has been determined to have thalamic damage at baseline.

52. A pharmaceutical composition comprising an amount of laquinimod for use in inhibiting or reducing thalamic damage in a human patient who has been determined to have thalamic damage at baseline.

53. Laquinimod for use in inhibiting or reducing thalamic damage in a subject afflicted with a disease or disorder other than a form of multiple sclerosis or a clinically isolated syndrome.

54. A pharmaceutical composition comprising an amount of laquinimod for use in inhibiting or reducing thalamic damage in a subject afflicted with a disease or disorder other than a form of multiple sclerosis or a clinically isolated syndrome.

55. Laquinimod for use in inhibiting or reducing tremor or spasticity in a subject.

56. A pharmaceutical composition comprising an amount of laquinimod for use in inhibiting or reducing tremor or spasticity in a subject.
